(19) United States
(12) Patent Application Publication
(10) Pub. No.: US 2006/0275229 A1
(43) Pub. Date: Dec. 7, 2006

(54) Skin care active complex and methods of using same

(76) Inventors: Sreekumar Pillai, Wayne, NJ (US); Brian Costello, Lakegrove, NY (US); Christian Oresajo, Nanuet, NY (US); Joseph Ceccoli, Farmingville, NY (US)

Correspondence Address: Chief Patent Counsel;Engelhard Corporation, 101 Wood Avenue, P.O. Box 770, Iselin, NJ, 08830, US

(21) Appl. No.: 11/436,797

(22) Filed: May 17, 2006

Related U.S. Application Data
(60) Provisional application No. 60/594,908, filed on May 17, 2005.

(51) Int. Cl. A61K 8/34; A61K 8/64; A61K 8/97

(52) U.S. Cl. 424/59; 424/70.14; 424/70.1; 424/74

(57) ABSTRACT

A skin care active complex in accordance with an embodiment of the invention comprises a combination of (1) from about 0.001% to about 10% by weight, preferably from about 0.001% to about 5% by weight, of a vitamin A derivative; (2) from about 0.00001% to about 10% by weight of a hydroxamate derivative; (3) from about 0.01% to about 5% by weight of an anti-inflammatory natural compound; and optionally, (1) from about 0.001% to about 10% by weight of vitamin K.

### FIELD OF THE INVENTION

The present invention is directed to a skin care composition, also referred to herein as a “skin care active complex”, and a method for using same, particularly to prevent or retard the process of cutaneous aging.

### BACKGROUND OF THE INVENTION

Photoaging is the change in appearance and/or function of human skin as a result of repeated exposure to sunlight, resulting in wrinkles and other changes in the appearance of the skin.

Solar radiation reaching the earth's surface both effects and enables various animals, including humans. This solar radiation comprises ultraviolet (UV) (lamda<400 nm), visible (400 nm<lamda<700 nm), and infrared (IR) (lamda>700 nm). UV radiation is generally divided into UVA (320-400 nm), UVB (290-320 nm), and UVC (<290 nm). The ultraviolet (UV) component of sunlight, particularly UVB, is generally believed to be the principal causative agent in photoaging.

The extent of UV exposure required to cause photoaging is not currently known, although the amount required to cause erythema (reddening, commonly seen as sunburn) in human skin is known and quantified empirically as the “minimal erythemal dose” (“MED”) from a given UV source. UVB wavelengths of 290-300 nm are the most erythmogenic. The effectiveness of UV radiation in causing erythema decreases rapidly as the UV wavelength is increased beyond about 300 nm; wavelengths of 320 nm and 340 nm are, respectively, one hundred and one thousand times less potent at causing skin reddening than wavelengths of about 298 nm. Repeated exposure to sunlight at levels that cause erythema and tanning are, nevertheless, commonly associated with photoaging.

Photoaging in human skin is characterized clinically by coarseness, wrinkles, mottled pigmentation, sallowness, laxity, eventually premalignant, and ultimately malignant neoplasm. Photoaging commonly occurs in skin that is habitually exposed to sunlight, such as the face, ears, bald areas of the scalp, neck, forearms, and hands. Various attempts have been made to address the damage to the skin caused by photoaging.

Sunscreens are commonly used to prevent photoaging of skin areas that are exposed to sunlight. Sunscreens are topical preparations that contain ingredients that absorb, reflect, and/or scatter UV light. Some sunscreens are based on opaque particulate materials including zinc oxide, titanium oxide, clays, and ferric chloride. Because such preparations are visible and occlusive, many people consider these opaque formulations cosmetically unacceptable.

Other sunscreens contain chemicals such a p-aminobenzoic acid (PABA), oxybenzone, dioxybenzone, ethylhexyl-methoxy cinnamate, octocrylene, octyl methoxycinnamate, and butylmethoxydibenzoylmethane that are transparent or translucent on the skin. While these types of sunscreens may be more acceptable cosmetically, they are still relatively short-lived and susceptible to being removed by washing or perspiration.

The generally accepted etiology of photodamage to skin involves an exposure to sunlight sufficient to cause erythema (sunburn or reddening; literally a flush upon the skin), and it is now known that sufficient UVB radiation does cause erythema. This philosophy dictates that present compositions and methods for inhibiting photoaging include the use of compounds that block or absorb UVB, and that such compositions need be used only when there is sufficient likelihood that exposure to sunlight will result in erythema. More recent sunscreen compositions include combinations of compounds that block both UVA and UVB radiation.

Retinoids have also been used as therapy to improve the appearance of sun-damaged skin. In particular, U.S. Pat. No. 4,877,805 discloses the treatment of photoaged skin. The patent indicates that there is little point in beginning the application of a retinoid to treat photodamage until the effects of aging begin to appear.

Matrix metalloproteinases (MMPs) are a family of enzymes that play a major role in physiological and pathological destruction of connective tissue, especially collagen. Various types of collagen and collagenases (types of MMPs) are known in this field. U.S. Patent Application Ser. Nos. U.S. 2002/0010162A1 and U.S. 2002/0198176A1 disclose treatment of psoriasis with MMP inhibitors. It is believed that chronological age as well as UV induction stimulates the production of MMP levels in the skin. This causes degradation of skin collagens and other matrix components, resulting in thinning of the dermis and eventual wrinkle formation. Photoaging can be inhibited by inhibiting the MMP activity using specific MMP inhibitors in combination with retinol or other vitamin A analogues to stimulate collagen syntheses and inhibits the UV induced mechanism leading to the synthesis of MMP proteins. U.S. Pat. No. 6,130,254 discloses methods for inhibiting photoaging of skin using retinoid and an MMP inhibitor. U.S. Pat. No. 6,630,516 discloses methods and compositions for preventing and treating chronological aging in human skin using retinoid and an MMP inhibitor. U.S. Pat. No. 6,682,763 discloses skin-beautifying agent, anti-aging agent for the skin, whitening agent and external agent for the skin using extracts prepared from olive plants.

Often individuals are also concerned with reducing puffiness and discoloration under the eyes as well as lessening the appearance of wrinkles due to photoaging. Separately, Vitamin K has been demonstrated to be effective in reducing bruising following certain dermatologic procedures. Vitamin K has been used for treatment of blood vessel disorders of the skin including purpura, rosacea and vascular problems of subcutaneous tissues. U.S. Pat. No. 5,510,391 discloses a method of treating blood vessel disorder of the skin using vitamin K.

### SUMMARY OF THE INVENTION

According to the present invention, there are provided various embodiments of a skin care active complex and cosmetic products comprising such complexes. Also provided are methods for preventing or retarding the process of cutaneous aging that involve decreased collagen synthesis and its increased breakdown, increased capillary fragility and permeability.

In one embodiment, the complex includes an MMP inhibitor to prevent skin collagen breakdown with age and UV radiation, retinol for boosting collagen synthesis and stimulating epidermal cell proliferation, and ursolic acid as an anti-inflammatory agent and for stimulating epidermal differentiation. In another embodiment of the invention particularly useful in cosmetic products targeting the eye area, the complex further includes vitamin K for strengthening capillaries and decreasing skin bruising. However, other embodiments of the invention without vitamin K are particularly useful in cosmetic products that are not concerned with under eye discoloration or puffiness, or for cosmetic products sold in geographic regions where the use of vitamin K may be regulated.

The methods involve topically applying a complex according to the invention in the facial area, and optionally around the eyes, to prevent both UV induced and age-induced wrinkle formation, improve general skin condition, reducing dark circles around the eye area, and for the treatment of skin redness and rosacea. This combination of ingredients helps to strengthen capillaries, decrease capillary fragility and permeability, and stimulate blood flow, in combination with powerful anti-inflammatory activities. In addition, the ingredients help stimulate collagen synthesis, and inhibit collagen and elastin breakdown.

One embodiment of this invention is a skin care active complex comprising one or more vitamin A derivatives in an amount of from about 0.001% to about 10% by weight; one or more hydroxamate derivatives in an amount of from about 0.00001% to about 10% by weight; one or more anti-inflammatory natural compound in an amount of from about 0.01% to about 5% by weight, and optionally, one or more vitamin K in an amount of from about 0.001% to about 10% by weight.

Another embodiment of this invention is a cosmetic product comprising from about 1 to about 2% by weight of a skin care active complex that comprises one or more vitamin A derivatives in an amount of from about 0.01% to about 10.0% by weight, preferably 0.01% to about 5.0% by weight, and more preferably 0.01% to about 1.0% by weight; one or more hydroxamate derivatives in an amount of from about 0.0001% to about 0.01% by weight; one or more anti-inflammatory natural compound in an amount of from about 0.1% to about 1% by weight, and optionally, one or more vitamin K in an amount of from about 0.01% to about 0.1% by weight.

Another embodiment of this invention is a sunscreen product comprising from about 1 to about 2% by weight of a skin care active complex that comprises one or more vitamin A derivatives in an amount of from about 0.01% to about 10.0% by weight, preferably 0.01% to about 5.0% by weight, and more preferably 0.01% to about 1.0% by weight; one or more hydroxamate derivatives in an amount of from about 0.0001% to about 0.01% by weight; and one or more anti-inflammatory natural compound in an amount of from about 0.1% to about 1% by weight, optionally, one or more vitamin K in an amount of from about 0.01% to about 0.1% by weight; and an effective amount of a sunscreen agent.

Another embodiment of this invention is a method of preventing UV-induced and age-induced wrinkle formation comprising formulating a skin care active complex that comprises vitamin A derivatives in an amount of from about 0.001% to about 5% by weight; hydroxamate derivatives in an amount of from about 0.00001% to about 10% by weight; anti-inflammatory natural compound in an amount of from about 0.01% to about 5% by weight, and optionally, one or more vitamin K in an amount of from about 0.001% to about 10% by weight.

Another embodiment of this invention is a method of improving skin condition comprising formulating the above active complex, and applying the complex on the skin in one of a cream, lotion and a wash off type product.

Another embodiment of this invention is a method of brightening skin comprising formulating the above active complex, and applying the composition on the skin in one of a cream, lotion and a wash off type product.

Another embodiment of this invention is a method of treating rosacea and redness in skin, comprising formulating the above composition, preferably comprising one or more vitamin K, and applying the composition on the skin in one of a cream, lotion and a wash off type product.

Another embodiment of this invention is a method of manufacturing a skin care active complex comprising combining one or more vitamin A derivatives in an amount of from about 0.01% to about 10.0% by weight, preferably 0.01% to about 5.0% by weight, and more preferably 0.01% to about 1.0% by weight, one or more hydroxamate derivatives in an amount of from about 0.00001% to about 10% by weight; one or more anti-inflammatory natural compound in an amount of from about 0.01% to about 5% by weight, and optionally, one or more vitamin K in an amount of from about 0.001% to about 10% by weight.

### DETAILED DESCRIPTION OF THE PREFERRED EMBODIMENTS

The present invention as described herein is directed to a skin care active complex made up of a combination of vitamin A derivatives, hydroxamate derivatives anti-inflammatory natural compounds, and optionally, vitamin K. In a preferred embodiment, the vitamin A derivative is retinol, the hydroxamate derivative is pesudopeptide hydroxamate, the anti-inflammatory natural compound is ursolic acid, and vitamin K, in the form of vitamin K-1, is utilized.

This combination (hereinafter referred to as “antiaging complex” in the examples) can form an active complex with capillary strengthening, anti-inflammatory, collagen-boosting and matrix metalloprotease inhibitory activities.

Each component of the skin care active complex in this invention provides a particular function. Specifically, retinol can provide anti-aging and collagen boosting to improve mottled pigmentation, fine lines and wrinkles, skin texture, skin tone and color, and skin's hydration levels. Pseudopeptide hydroxamate, a strong zinc chelator and a matrix metalloprotease inhibitor, can prevent inflammation, block matrix degradation, increase dermal collagen content and provide anti-aging benefits. Ursolic acid can prevent inflammation and can block age- and sun-induced matrix breakdown by inhibiting elastase activity. Optionally, vitamin K can reduce skin bruising, stimulates blood circulation and decrease skin discoloration, especially around the eye area. In the interest of clarity, the features of an actual implementation for each component, which are individually well known to those of skill in the art, are described in detail herein.

Vitamin A Derivatives

Retinol, a derivative of vitamin A, is a safer alternative to retinoic acid, and is the most active vitamin A derivative in the skin. Retinol has a molecular structure that helps to penetrate into the lower layers of skin, where it can stimulate the production of collagen and elastin. Retinol can improve mottled pigmentation, fine lines and wrinkles, skin texture, skin tone and color, and skin's hydration levels. Retinol can be converted to retinoic acid within the upper layers of the skin and can stimulate various processes involved in epidermal turnover, dermal matrix synthesis and stimulation of skin repair processes. All these activities result in healthier and younger looking skin. Retinol and its natural and synthetic derivatives (commonly called retinoids) are used extensively for the treatment of a variety of skin conditions.

Both retinol and its derivatives have been used as pharmacologic agents to treat disorders of the skin. In an embodiment of this invention, the vitamin A derivative is retinol. Another embodiment of the present invention includes use of retinol esters with saturated or unsaturated fatty acids (mono- or di-unsaturated) of chain length from 8-20.

Hydroxamate MMP Inhibitors

Pseudopeptide hydroxamate, also known as ilomastat or Galardin (or GM 6001), has been designed to mimic the substrate of matrix metalloproteases (MMP) such that it can lie within the cleft of the active site and bind the zinc atom within the active site, rendering the metalloprotease inactive. The chemical group that interacts with the zinc atom is a hydroxamate group. The isobutyl group and the tryptophan side chain of the molecule mimic the substrate and bind to the active site of the MMP. This allows a tight but reversible binding of the inhibitor to the enzyme active site.

Pseudopeptide hydroxamate is a topically active molecule, which has been demonstrated to have anti-inflammatory activity. It has been tested in clinical trials for treatment of corneal ulceration. Its safety in corneal and topical preparations has been validated in phase 1 and 2 clinical trials. The usefulness of this molecule for treatment of inflammatory skin disorders, inhibition of angiogenesis, wound healing, strengthening of wounds and for prevention of scar formation has been documented.

In an embodiment of this invention, the hydroxamate derivative is pseudopeptide hydroxamate.

Other embodiments of the present invention include other hydroxamate derivatives including, but not limited to, salicylic acid and its derivatives, hydroxybenzoic acid and other heterocyclic compounds, tryptophan, amino acids and small peptides (peptides containing from 1 to 6 amino acids), peptide mimetics, alpha hydroxy acids and their derivatives, and dicarboxylic acids, are useful. Other useful hydroxamates include hydroxamate derivatives of substrate-analogue peptides of matrix metalloproteases containing aminomalonic acid, and monohydroxamates of aspartic and glutamic acids.

Anti-Inflammatory Natural Compounds

Ursolic acid is a known inhibitor of human leucocyte elastase (HLE).

Elastin is the protein that gives body tissues their elasticity, and imparts youthful suppleness and resilience to the skin. Elastin fibers help skin to maintain its tensile strength as well as the ability to recoil after being stretched.

Maintaining adequate levels of elastin can diminish the effects of aging. Elastase is a protease that specifically degrades elastin. It is always present in skin at low levels where it is needed for growth, tissue repair and the natural slow elastin turnover that takes place in healthy skin. Elastase is also a component of the defense system, and when skin is inflamed, levels of elastase increase dramatically. The release of elastase during the natural cellular processes, as well as during the aging process, contributes to the loss of skin tone over time. As skin ages, the level of elastase increases to a point where elastin breaks down faster than it can be made. This results in the dramatic loss of skin elasticity. Several pentacyclic triterpenoid metabolites of plant origin, including ursolic acid, are natural inhibitors of elastase. Application of these naturally occurring compounds in vitro has shown them to be very effective elastase inhibitors, combating age-related changes as well as inflammation.

Ursolic acid treatment can improve the health of skin and hair. Ursolic acid and its derivatives can form oil-resistant barriers on the skin and hair similar to the waxy coating of fruits. Ursolic acid has been used to treat photoaged skin because it can prevent and improve the appearance of wrinkles and age spots by restoring the skin's collagen bundle structures and its elasticity. Ursolic acid has also been reported to increase ceramide synthesis in skin cells and improve the barrier properties of skin. Ursolic acid is also a potent anti-inflammatory agent due to its powerful inhibitory effect on 5-lipoxygenase and cyclooxygenase activity.

In an embodiment of this invention, the anti-inflammatory natural compound is ursolic acid.

Other natural anti-inflammatory compounds include natural vitamins and plant extracts. Most of the anti-oxidant compounds also have anti-inflammatory activities. Therefore, a list of anti-inflammatory compounds also includes natural antioxidants. Useful anti-oxidants include, but are not limited to, vitamin E (tocopherol) and its esters, vitamin C and its derivatives, green tea polyphenols, coenzyme Q10, quercetin and other bioflavinoids, plant extracts such as grape seed, pomegranate, amla (emblica Officinale), and other polyphenolic antioxidants such as nordihydroguaiaretic acid (NDGA).

Commonly used antioxidants useful for skin care include, but are not limited to, vitamins such as vitamin C and its derivatives, vitamin E (tocopherol) and its esters, ubiquinone, coenzyme Q10 and lipoic acid.

Polyphenolic antioxidants also include caffeic acid, ferulic acid, quercetin, apigenin, genistein, resveratrol, nordihydroguaiaretic acid, carnosic acid, ursolic acid, rosemarinic acid, silymarin, epicatechin, epicatechin-3-gallate, epigallocatechin, epigallocatechin gallate, procyanidins, proanthocyanidins, gallotannins, ellagotannis and pycnogenol.

In addition, a variety of plant-derived compounds have excellent antioxidant activities. These include flavonoids and polyphenols. Flavonoids are polyphenolic compounds that are ubiquitous in nature and are categorized, according to chemical structure, into flavonols, flavones, flavanones, isoflavones, catechins, anthocyanidins and chalcones. Over 4,000 flavonoids have been identified, many of which occur in fruits, vegetables and beverages such as tea, coffee, beer, wine and fruit drinks.

Vitamin K

Vitamin K plays an important role in blood clotting. However, it has been recently discovered that vitamin K can be used for the treatment of dark circles under the eyes and bruising on the face. When the fat pad beneath the eye begins to thin with age, it can create a sunken look to the under-eye area. Studies have shown that sluggishness of blood flow underneath the eyes may also contribute to dark circles. Vitamin K has been found to diminish the appearance of these dark circles. Vitamin K has also recently been demonstrated to be effective in reducing bruising following certain dermatologic procedures. In one study, patients underwent laser treatments to lessen the appearance of spider veins on the face. Since the laser treatment may cause bruising, half the patients applied topical vitamin K to half their faces for two weeks before laser treatment and a placebo cream to the other half of their face. The remaining patients applied the vitamin K to one half of the face and the placebo to the other half, after treatment. While the application of topical vitamin K before the procedure did not seem to affect the severity of bruising, those patients who applied the vitamin K after the procedure noticed a significant reduction in the severity of bruising.

Vitamin K is found in the natural form of vitamin K-1 (also known as phytonadione and produced by green leafy vegetables) and vitamin K-2 (produced by gastrointestinal bacteria). In addition, vitamin K-analogs have been synthesized and currently include vitamins K-3, K-4, K-5, K-6 and K-7.

In an embodiment of this invention, the vitamin K is vitamin K-1. The phrase “one or more vitamin K” as used herein means one or more of a natural form or analog of vitamin K.

Preferably, the skin care active complex further comprises one or more solvents. Examples of suitable solvents include, but are not limited to ethoxydiglycol, octyldodecanol, butylene glycol, propylene glycol, and caprylic/capric triglyceride.

A skin care active complex according to the invention may be utilized in a cosmetic product in a concentration of about from about 1 to about 2% by weight. This composition can be used in anti-aging, anti-wrinkle and moisturizing skin care products for all types of skin (dry, normal, and sensitive). It can be incorporated in post-sun products, and is particularly useful for the treatment of mature (over age 55) skin. It may also be used in make-up products. The active complex can be incorporated in various types of product formulations, including but not limited to cream, lotion and wash-off products. Preferably, the active complex is incorporated into a product formulation at a pH range of 4.5-7.5.

A sunscreen product incorporating a composition of an embodiment of the invention and an effective amount of sunscreen agent can be formed. Sunscreen agents include those materials commonly employed to block ultraviolet light. Illustrative compounds are the derivatives of PABA, cinnamate and salicylate. For example, octyl methoxycinnamate and 2-hydroxy-4-methoxy benzophenone (oxybenzone) can be used. Octyl methoxycinnamate and 2-hydroxy-4-methxy benzophenone are commercially available under the tradenames Parsol MCX and Benzophenone-3, respectively. The exact amount of sunscreen agents employed in the emulsions can vary depending upon the degree of protection desired from the ultraviolet radiation.

### EXAMPLES

### Example 1

### Inhibition of Lipid Peroxidation by the Anti-Aging Complex

(Antioxidant Test)

The antioxidant capacity of the anti-aging complex of the present invention was tested by measuring its ability to prevent UV-induced peroxidation of phospholipids. Unsaturated fatty acids are present in the phospholipids of cell membranes. Their oxidation compromises membrane integrity and also leads to the generation of breakdown products that are damaging to other important cellular components such as proteins and nucleic acids. Since the skin is exposed to large doses of UV light from the sun, UV-induced peroxidation is a significant route of oxidant damage.

Antioxidant activity was assessed by exposing mixtures of phospholipid liposomes to UV light in the presence of varying concentrations of the anti-aging complex. The rate of oxidative damage was determined by the increase in the levels of malonaldehyde (a breakdown product of oxidized lipids) as a function of UV exposure time.

TABLE 1
Inhibition of UV-Induced Peroxidation by Anti-aging Complex

| Concentration of Anti-aging Complex | % Inhibition |
|---|---|
| (% v/v) | Lipid Peroxidation |
| 0.03 | 8 ± 2 |
| 0.1 | 82 ± 2 |

The results are shown in Table 1 above. The left column of Table 1 lists the concentration of the anti-aging complex that was tested, while the right column lists the amount of inhibition of lipid peroxidation. It can be shown that a concentration of 0.03% v/v of the anti-aging complex inhibited lipid peroxidation only by about 8±2%, while a concentration of 0.1% v/v of the anti-aging complex produced an inhibition of lipid peroxidation of about 82±2%. Based on this data, the EC50 value (concentration that inhibits oxidation by 50%) was calculated to be about 0.07% v/v. Accordingly, a material with this level of potency is considered to be a good antioxidant with respect to UV-induced change to the unsaturated phospholipids that are present in skin cell membranes.

### Example 2

### Comparison of Common Antioxidants

The ability of an anti-aging complex in accordance with an embodiment of the invention to prevent UV-induced lipid peroxidation was measured in comparison to other antioxidant industry standards such as vitamin E, vitamin C, and Epigallocatechin gallate (EGCG). The results are displayed in the Table 2 below.

TABLE 2
Activity of Common Antioxidants

| | EC50 (concentration that inhibits |
|---|---|
| Antioxidant | oxidation by 50%) (% w/w) |
| Epigallocatechin gallate (EGCG) | 0.0003 |
| Vitamin C (Ascorbic Acid) | 0.002 |
| Vitamin E acetate | 0.022 |
| Anti-aging Complex | 0.07 |

The left column of Table 2 lists the compounds that were tested, while the right column lists each corresponding compound the effective concentration that inhibits oxidation by 50%. The assay was performed by irradiating a suspension of lecithin liposomes with UV light for 4 hours in the absence of or in the presence of the different compounds listed in the table. Malonaldehyde levels, a breakdown product of oxidized lipids, were measured in aliquots of the reaction mixture at different times. The rate of lipid oxidation was calculated from the increase in malonaldehyde content. As can be seen from Table 2, the results demonstrate that an anti-aging complex in accordance with an embodiment of the invention possesses antioxidant activity almost as good as pure vitamin E.

### Example 3

### Inhibition of Matrix Degrading Proteases by the Antiaging Complex

The anti-aging complex in accordance with an embodiment of the present invention was tested for its ability to inhibit matrix metalloproteinase-1 (MMP-1; also called fibroblast collagenase), MMP-9 (Gelatinase B) and Neutrophil Elastase. In combination, these three enzymes are capable of extensively degrading all of the protein components of the extracellular matrix (ECM) including collagens I and III (the major collagen types in the skin), elastin and the various glycoprotein components (e.g., fibronectin). Elevated expression of these proteases is triggered by inflammatory stimuli such as UV light, and their activity results in ECM damage that is a major component of skin aging.

The results of these tests are summarized in the tables below. The anti-aging complex is shown to possess activity to be moderate to good protease inhibitors.

### Example 3A

### MMP-1 Inhibitory Activity of Antiaging Complex

TABLE 3
Inhibition of MMP-1 by Anti-Aging Complex

| Concentration of Anti-Aging Complex | % Inhibition |
|---|---|
| (% v/v) | of MMP-1 |
| 0.1 | 8 ± 2 |
| 0.3 | 44 ± 4 |
| 1 | 90 ± 0.5 |
| 2 | 95 ± 0.5 |

Table 3 above shows that the anti-aging complex demonstrates significant inhibition to MMP-1. Table 3 provides a tabulation of the concentration of anti-aging complex (in % v/v) in the left column and the corresponding level of MMP-1 inhibition (in %) based on the anti-aging complex in the right column. A composition containing about 0.1% v/v anti-aging complex is shown to have an about 8±2% inhibition of MMP-1. A composition containing about 0.3% v/v anti-aging complex is shown to have an about 44±4% inhibition of MMP-1. A composition containing about 1% v/v anti-aging complex is shown to have an about 90±0.5% inhibition of MMP-1. A composition containing about 2% v/v anti-aging complex is shown to have an about 95±0.5% inhibition of MMP-1.

### Example 3B

### MMP-9 Inhibitory Activity of Antiaging Complex

TABLE 4
Inhibition of MMP-9 by Anti-Aging Complex

| Concentration of Anti-Aging Complex | % Inhibition |
|---|---|
| (% v/v) | of MMP-9 |
| 0.03 | 25 ± 1.6 |
| 0.1 | 53 ± 2.2 |
| 0.3 | 85 ± 0.8 |
| 1 | 94 ± 0.6 |

Table 4 above shows that the anti-aging complex demonstrates significant inhibition to MMP-9. Table 4 provides a tabulation of the concentration of anti-aging complex (in % v/v) in the left column and the corresponding level of MMP-9 inhibition (in %) based on the anti-aging complex in the right column. A composition containing about 0.03% v/v anti-aging complex is shown to have an about 25±1.6% inhibition of MMP-9. A composition containing about 0.1% v/v anti-aging complex is shown to have an about 53±2.2% inhibition of MMP-9. A composition containing about 0.3% v/v anti-aging complex is shown to have an about 85±0.8% inhibition of MMP-9. A composition containing about 1% v/v anti-aging complex is shown to have an about 94±0.6% inhibition of MMP-9.

### Example 3C

### Neutrophil Elastase Inhibition by Antiaging Complex

TABLE 5
Inhibition of Neutrophil Elastase by Anti-Aging Complex

| Concentration of Anti-Aging Complex | % Inhibition of |
|---|---|
| (% v/v) | Neutrophil Elastase |
| 0.03 | 10 ± 0.05 |
| 0.06 | 15 ± 0.05 |
| 0.125 | 24 ± 1.2 |
| 0.25 | 43 ± 0.8 |
| 0.5 | 65 ± 2.6 |
| 1 | 82 ± 8.9 |
| 2 | 100 ± 0.8 |

Table 5 above shows that the anti-aging complex demonstrates significant inhibition to Neutrophil Elastase. Table 5 provides a tabulation of the concentration of anti-aging complex (in % v/v) in the left column and the corresponding level of Neutrophil Elastase inhibition (in % v/v) based on the anti-aging complex in the right column. A composition containing about 0.03% v/v anti-aging complex is shown to have an about 10±0.05% inhibition of Neutrophil Elastase. A composition containing about 0.06% v/v anti-aging complex is shown to have an about 15±0.05% inhibition of Neutrophil Elastase. A composition containing about 0.125% v/v anti-aging complex is shown to have an about 24±1.2% inhibition of Neutrophil Elastase. A composition containing about 0.25% v/v anti-aging complex is shown to have an about 43±0.8% inhibition of Neutrophil Elastase. A composition containing about 0.5% v/v anti-aging complex is shown to have an about 65±2.6% inhibition of Neutrophil Elastase. A composition containing about 1% v/v anti-aging complex is shown to have an about 82±8.9% inhibition of Neutrophil Elastase. A composition containing about 2% v/v anti-aging complex is shown to have an about 100% inhibition of Neutrophil Elastase.

### Example 3D

### IC50 Values for Protease Inhibition

The IC50 is the concentration of test sample that inhibits protease activity by 50%, a standard measure of potency. A lower value for the IC50 indicates a more potent inhibitor. The IC50 values as determined from the experiments shown in Tables 3 to 5 are shown in Table 6 below.

TABLE 6
IC50 Values for Protease Inhibition by the Anti-Aging Complex

| Enzyme | IC50 (% v/v) |
|---|---|
| MMP-1 | 0.4 |
| MMP-9 | 0.08 |
| Neutrophil Elastase | 0.34 |

Table 6 above tabulates the IC50 values for protease inhibition by enzymes used to study the ability of the anti-aging complex to inhibit MMP-1, MMP-9 and Neutrophil Elastase enzymes. In Table 6, the left column provides a list of the enzymes tested and the right column provides the IC50 values (in % v/v) corresponding to the enzymes. Here, the IC50 (% v/v) for MMP-1 inhibition is about 0.4, for MMP-9 is about 0.08 and for Neutrophil Elastase is about 0.34.

The in vitro study data shows that the anti-aging complex is capable of providing significant anti-aging benefits by inhibiting proteases that degrade the extracellular matrix in skin.

### Example 4

### Elastase Inhibition Activity of Ursolic Acid, a Component of the Antiaging Complex

Ursolic acid is a powerful inhibitor of elastase activity. The inhibition activity of ursolic acid was tested by incubating it with a human neutrophil elastase sample (obtained from Athens Research and Technology, GA) for 10 minutes with a chromogenic substrate, given below. The change in optical density of the substrate at 405 nm was recorded one reading per minute at room temperature reaction mixtures in a 96-well plate. The reaction mixture contains 300 g/ml methoxysuccinyl-Ala-Ala-Pro-Val-pNA (chromogenic substrate); 1.3 g/ml human neutrophil elastase at concentrations in the range 0.55%-3.3%; 70 mM Tris-HCl (pH 8); and 200 mM NaCl.

The % inhibition is calculated relative to the no-sample control. The results are displayed in a graph which is tabulated in Table 7 below, in which the percent (%) elastase inhibition activity of ursolic acid is plotted versus the concentration of ursolic acid.

TABLE 7
Elastase Assay: Ursolic Acid

| Concentration (%) | Inhibition (%) |
|---|---|
| 0 | 0 |
| 0.0004 | 26 |
| 0.0008 | 44 |
| 0.0012 | 58 |
| 0.002 | 72 |
| 0.004 | 81 |
| 0.006 | 84 |
| 0.01 | 86 |

Table 7 above indicates that the IC50 value for elastase inhibition for ursolic acid is about 0.0008%. When a composition (or anti-aging complex) according to an embodiment of the present invention was tested in this assay, the IC50 value for the anti-aging complex was 0.34% as shown in Table 6, in agreement with the ursolic acid content of the product.

### Example 5

### Differentiation of Epidermal Keratinocytes

The anti-aging complex was tested for its ability to promote a less differentiated and more proliferative state of keratinocyte metabolism. Such an effect would be expected to promote skin renewal and contribute to an anti-aging benefit. For this test, epidermal keratinocytes were treated with varying concentrations of anti-aging complex for several days in the presence of a high concentration of calcium. A high calcium level is characteristic of the outer layers of the epidermis and is conducive to keratinocyte differentiation as opposed to proliferation. At the end of the treatment period the cell cultures were solubilized with detergent and the content of insoluble cornified envelopes (CE) was determined by optical diffraction. Cornified envelopes are formed during terminal differentiation of keratinocytes as their proteins become covalently cross-linked. This cross-linking forms squames that are the “bricks” in the brick-and-mortar barrier of the stratum corneum.

The effect of anti-aging complex is shown in the Table 8 below where the CE content is calculated as a percentage of the content in untreated cultures.

TABLE 8
Effect of Anti-aging Complex on Cornified Envelop Content
(terminal differentiation) of Cultured Epidermal Keratinocytes

| Anti-aging Complex | CE Content | CE Content |
|---|---|---|
| Concentration (% v/v) | (% of Control) | (% Inhibition) |
| 0 (control) | 100 | 0 |
| 0.00006 | 53 | 47 |
| 0.0002 | 38 | 62 |
| 0.0006 | 28 | 72 |

The left column of Table 8 lists the anti-aging complex in three different concentrations (compared to control in the top row) as measured in % v/v. The middle column provides the cornified envelop formation as percent of control (taken as 100%). The right column provides the percent inhibition of the specific cornified envelop content corresponding to the anti-aging complex. The results of this study shows that a concentration of 0.00006% v/v anti-aging complex correlates to an inhibition to CE content of about 47%. The result also show that a concentration of 0.0002% v/v anti-aging complex correlate to an inhibition to CE content of about 62%. Further, the results show that a concentration of 0.0006% v/v of the anti-aging complex correlate to an inhibition to CE content of about 72%. At concentrations as low as 0.00006% v/v, anti-aging complex inhibited CE formation by approximately 50%. This is potent activity that would be expected to contribute to an anti-aging effect on the skin.

It can be shown that the anti-aging complex supports anti-aging benefit by promoting a less differentiated and therefore, a more regenerative phenotype in epidermal keratinocyte, the effect of which is conducive to skin renewal.

### Example 6

### Epidermal Penetration of Pseudopeptide Hydroxamate from the Anti-Aging Complex

Studies were conducted to determine the effect of the anti-aging complex formulation on epidermal penetration of pseudopeptide hydroxamate (PPH), which is the MMP inhibitor in at least one embodiment of the present invention. These studies were conducted on EpiDerm tissues (MatTek Corporation) by applying test sample (the “donor”) to the upper surface (stratum corneum) and measuring the concentration of PPH in the “receptor” fluid (growth medium) below the tissue at time points over a 24 hour period. The anti-aging complex was compared to a conventional 1000 ppm solution of PPH in butylene glycol (BG). For these studies the PPH content of the anti-aging complex was supplemented to a final concentration of 80 ppm in order to ensure that the concentration of penetrated PPH in the receptor chamber would be within the range of our analytical HPLC method (which is less sensitive than the MMP inhibition tests).

TABLE 9
Benefit of Anti-aging Complex Formulation for Pseudopeptide
Hydroxamate Penetration

| Product | Donor/Receptor Ratio |
|---|---|
| BG Solution | 19,542 |
| Anti-aging Complex | 687 |

At the end of the 24-hour incubation period, the mean PPH concentrations in the receptor fluids were 0.0535 ppm for the BG solution and 0.1165 ppm for the anti-aging complex. In Table 9 above, the left column provides the product tested, specifically, BG solution and the anti-aging complex, while the right column provides the resulting tabulation of the donor/receptor ratio. Expressing these concentrations in a donor/receptor concentration ratio yields values of about 687 for the anti-aging complex and about 19,542 for the BG solution, as shown in the table above. Hence, the donor/receptor ratio is about 28× greater for the BG solution compared to the anti-aging complex, despite the fact that PPH was 12.5× more concentrated in the BG solution compared to the anti-aging complex. This indicates that PPH penetrated much more efficiently when applied along with the anti-aging complex. Hence the anti-aging complex formulation provides significant benefit with regard to delivery of this active.

The anti-aging complex formulation is demonstrably superior to a conventional formulation with respect to delivering pseudopeptide hydroxamate, the main MMP inhibiting component.

### Example 7

### Clinical Study

To measure the in vivo efficacy of an anti-aging complex in accordance with an embodiment of the invention, 52 subjects participated in a study. After a 4-day wash-out period, subjects came to a test center and equilibrated to room conditions for 15 minutes. Baseline instrumental measurements were taken. Subjects were instructed to use the test product twice daily for six (6) weeks. After four (4) weeks of product application, subjects returned for instrumental evaluations and were given fresh product to use for the remaining two (2) weeks. After six (6) weeks of product application, subjects returned for a final set of measurements. Changes from baseline were determined. The results are presented in Table 10 below.

TABLE 10
Effectiveness of Anti-aging Complex

| | 4 Weeks | 6 Weeks |
|---|---|---|
| Increase in Skin Moisturization | 6% | 14% |
| Improvement in Skin Tone | 2% | 3% |
| Improvement in Under-Eye Dark Circle | 25% | 26% |
| Improvement in Fine Lines & Wrinkles | 24% | 24% |

The left column of Table 10 lists those skin characteristics that were measured, while the middle and right columns record the measured improvement after 4 and 6 weeks, respectively. As can be seen from Table 10, clinical measurements taken at both four and six weeks show that the anti-aging complex in accordance with an embodiment of the invention increase skin moisturization, improve overall skin tone and under-eye dark circles, and decrease the appearance of fine lines and wrinkles.

A composition (or anti-aging complex) in accordance with an embodiment of the invention can be a powerful antioxidant at levels as low as about 0.07%, and can inhibit both matrix metalloproteases and skin elastases at low concentration levels (less than about 0.1%). In clinical studies, anti-aging complexes in accordance with an embodiment of the invention increased skin moisturization, improved skin tone, reduced under-eye circles and improved fine lines and wrinkles. These properties can make this product a unique ingredient for anti-aging, wrinkle reducing and under eye brightening ingredient in cosmetic formulations. Treatment with from about 1 to about 2% by weight of an anti-aging complex in accordance with an embodiment of the invention can prevent wrinkle formation, strengthens capillaries, reduces skin bruising stimulates blood flow and reduces puffiness and discoloration round the eye.

Accordingly, the composition in an embodiment of the present invention has shown to be effective in preventing UV-induced and age-induced wrinkle formation, improving skin condition, brightening skin, and/or treating rosacea and redness in skin.

### Example 8

### Manufacturing Procedure

For an anti-aging complex, ursolic acid (0.1 to 1.0%) was dissolved in ethoxydiclycol solvent by mixing the required amount of the ursolic acid in the solvent at room temperature with mixing. The required amount of retinol dissolved in caprylic/capric triglyceride was added to this mixture with mixing.

The final amount of retinol was between from 0.1 to 5% level and the caprylic/capric triglyceride level was between 5 to 20% in the final formulation.

Vitamin K-1 was added to the above mixture at a level of 0.01 to 0.1% level with mixing. A 1.0% stock solution of pesudopeptide hydroxamate was made in 100% propylene glycol. An amount of this stock solution was added to the formulation to obtain a final concentration of 0.0001% to 0.01% pseudopeptide hydroxamate in the final formulation. The formulation is then protected from oxidation by adding BHT at a level of less than 0.5%. This final formulation was stored in dark tight capped containers to protect the product from light and oxygen.

For a skin care active complex according to the present invention without vitamin K, ursolic acid (0.1 to 1.0%) was dissolved in octyldodecanol solvent by mixing the required amount of the ursolic acid in the solvent at room temperature with mixing. The required amount of retinol dissolved in caprylic/capric triglyceride was added to this mixture with mixing. The final amount of retinol was between from 0.1 to 10% level and the caprylic/capric triglyceride level was between 10 to 25% in the final formulation. A 1.0% stock solution of pesudopeptide hydroxamate was made in 100% propylene glycol. An amount of this stock solution was added to the formulation to obtain a final concentration of 0.0001% to 0.01% pseudopeptide hydroxamate in the final formulation. The formulation is then protected from oxidation by adding BHT at a level less than 1.0%. This final formulation was stored in dark tight capped containers to protect the product from light and oxygen.

### Example 9

### Skin Care Compositions

The following examples illustrate skin care active complexes according to the present invention. In one embodiment, the active complex is used as part of a cosmetic product, such as an anti-aging cosmetic product. A complex can be processed in one of the manners described in Example 8 above, or in a conventional manner suitable for cosmetic use. In particular, the complexes are suitable for applications to wrinkled, lined, rough, dry, flaky, aged and/or UV-damaged skin to improve the appearance and feel thereof as well as for application to healthy skin to prevent or retard deterioration thereof.

### Example 9A

This example is a skin care active complex according to an embodiment of the present invention. In an embodiment, the skin care active complex is used for preventing UV-induced and age-induced wrinkle formation, improving skin condition, brightening skin, and/or treating rosacea and redness in skin. In an embodiment, the complex is then incorporated in one of a cream, lotion, sunscreen and a wash off product. The complex comprises a combination of

(1) from about 0.001% to about 5% by weight of a vitamin A derivative;

(2) from about 0.00001% to about 10% by weight of hydroxamate derivative;

(3) from about 0.01% to about 5% by weight of an anti-inflammatory natural compound; and

(4) optionally, from about 0.001% to about 10% by weight of vitamin K.

In one embodiment, the vitamin K is vitamin K-1; the vitamin A derivative is retinol, the hydroxamate derivative is pseudopeptide hydroxamate, and the anti-inflammatory compound is ursolic acid.

### Example 9B

This example is a skin care active complex according to another embodiment of the present invention. The skin care active complex comprises a combination of (1) from about 0.01 to about 0.1% by weight of vitamin K-1; (2) from about 0.01 to about 1.0% by weight of retinol; (3) from about 0.0001% to about 0.01% by weight of pseudopeptide hydroxamate; and (4) from about 0.1 to about 1.0% by weight of ursolic acid.

### Example 10

The following examples illustrate cosmetic products according to the present invention. The products can be processed in a conventional manner as suitable for cosmetic use. In particular, the cosmetic products are suitable for application to wrinkled, lined, rough, dry, flaky, aged and/or UV-damaged skin to improve the appearance and the feel thereof as well as for application to healthy skin to prevent or retard deterioration thereof.

### Example 10A

This example illustrates an anti-aging cream incorporating a composition according to an embodiment of the present invention.

| Ingredients | % w/w |
|---|---|
| Antiaging complex | 2.0 |
| Butylene glycol | 3.0 |
| Disodium EDTA | 0.05 |
| Penulan TR2* | 0.1 |
| Carbowax PEG 400** | 5.0 |
| Montanov 68**** | 5.0 |
| Mineral Oil | 5.0 |
| Protachem CER*** | 5.0 |
| Preservative blend | 1.5 |
| Water | to 100 |

*from Noveon;
**from Dow Chemicals;
***from Protameen;
****from Seppic.

### Example 10B

This example illustrates an oil-in-water cream according to an embodiment of the present invention.

| Ingredients | % w/w |
|---|---|
| Antiaging complex | 2.0 |
| Mineral oil | 4.0 |
| 1,3-dimethyl-2- | 1.0 |
| imidazolidinone | |
| Alfol ® 16RD* | 4 |
| Triethanolamine | 0.75 |
| Butane-1,3-diol | 3 |
| Xanthan gum | 0.3 |
| Perfume | qs |
| Butylated hydroxy toluene | 0.01 |
| Water | to 100 |

*Alfol 16RD is cetyl alcohol and is a registered trademark of Condea Vista Co.

### Example 10C

This example illustrates a face rinse off lotion/gel composition according to the invention.

| Ingredients | % w/w |
|---|---|
| Antiaging complex | 2.0 |
| Antioxidant (vitamin C) | 2 |
| Butylene glycol | 5.0 |
| Preservative | 1.0 |
| Simulgel EPG | 2.5 |
| Biowax 754 liquid | 5.0 |
| Glycerin | 6.0 |
| Perfume | qs |
| Carbopol 980 | 0.5 |
| Water | to 100 |

### Example 10D

This example illustrates a composition for an anti-aging cream:

| Ingredients | % w/w |
|---|---|
| Butylene Glycol (1) | 5.00 |
| Glycerin (2) | 3.00 |
| Antiaging complex | 1.50 |
| Polymethylmethacrylate (Microma 100, | 2.00 |
| Ikeda) | |
| Cetearyl Alcohol and Polysorbate 60 and | 4.50 |
| PEG-150 Stearate and Steareth-20 | |
| Stearyl Heptanoate | 4.50 |
| Cetyl Recinoleate | 4.50 |
| Hydrogenated Polyisobutene | 3.00 |
| Isononyl Isononanoate | 1.30 |
| Synthetic Beeswax (Synthetic Beeswax | 1.75 |
| #122P, Koster Keunen) | |
| Hydroxyethyl Acrylate/Sodium | 1.00 |
| Acryloyldimethyl Taurate Copolymer and | |
| Squalane and Polysorbate 60 | |
| Cyclomethicone (DC345, Dow Corning) | 1.00 |
| Dimethicone (DC200/100, Dow Corning) | 0.2 |
| Tetrahexyldecyl Ascorbate | 0.25 |
| Preservative (Phenoxyethanol (and) | 1.20 |
| Chlorphenesin (and) Glycerin (and) | |
| Methylparaben (and) Benzoic Acid) | |
| Deionized Water | 1.5 |
| Ascorbyl Glucoside | 0.25 |
| Deionized Water | to 100 |

### Example 10E

This example illustrates an alcoholic lotion containing the inventive composition.

| Ingredients | % w/w |
|---|---|
| Antiaging Complex | 1.5 |
| 1,3-dimethyl-2- | 0.01 |
| imidazolidinone | |
| Ethanol | 40 |
| Antioxidant | 0.1 |
| Perfume | qs |
| Water | to 100 |

### Example 10F

This example illustrates a non-aqueous skin care composition incorporating the inventive combination.

| Ingredients | % w/w |
|---|---|
| Antiaging Complex | 2 |
| 1,3-dimethyl-2- | 1 |
| imidazolidinone | |
| Silicone gum SE-30 | 10 |
| Silicone fluid 345 | 20 |
| Silicone fluid 344 | 50.26 |
| Squalene | 10 |
| Linoleic acid | 0.01 |
| Cholesterol | 0.03 |
| 2-hydroxy-n-octanoic acid | 0.7 |
| Herbal oil | 0.5 |
| Ethanol | 2 |

### Example 10G

This example illustrates a sunscreen composition with SPF 15 incorporating the inventive combination.

| Ingredient | % w/w |
|---|---|
| Acrylates/C10-30 Alkyl Acrylate | 0.120 |
| Crosspolymer (Pemulen TR-2, | |
| Protameen) | |
| Antiaging complex | 1.0 |
| Butylene Glycol | 3.000 |
| Polymethylmethacrylate (Microma 100, | 1.000 |
| Kobo) | |
| Hydroxyethylacrylate/Sodium | 1.250 |
| Acryloyldimethyl Taurate Copolymer | |
| (and) Squalane (and) Polysorbate 60 | |
| (Simulgel NS, Seppic) | |
| Ethylhexyl Methoxycinnamate | 7.500 |
| Octyl Salicylate | 5.000 |
| Caprylic/Capric Triglyceride | 3.000 |
| Hydrogenated Polyisobutene | 5.000 |
| Pentaerythrityl Tetraisononanoate | 0.500 |
| (Pelemol P49, Phoenix) | |
| Acetylated Glycol Stearate (Cetacene, | 2.000 |
| Vevy) | |
| Cetearyl Alcohol (and) Ceteareth-20 | 3.000 |
| (Phoenoxol T, Phoenix) | |
| Octocrylene | 4.000 |
| Cetearyl Alcohol (and) Coco-Glucoside | 3.000 |
| Butyl Methoxydibenzoylmethane | 3.000 |
| Dimethicone (Silicone HL-88, Barnet) | 2.500 |
| Preservatives (Phenoxyethanol (and) | 1.250 |
| Chlorphenesin (and) Glycerin (and) | |
| Methylparaben (and) Benzoic Acid) | |
| Deionized water | to 100 |

It should be understood that the specific embodiments of the invention herein illustrated and described are intended to be representative only, as the invention may be modified and practiced in different but equivalent manners apparent to those skilled in the art having the benefit of the teachings herein. Furthermore, no limitations are intended to the details of composition herein shown, other than as described in the claims below. It is therefore evident that the particular embodiments disclosed above may be altered or modified and all such variations are considered within the scope and spirit of the invention. Accordingly, the protection sought herein is as set forth in the claims below.

What is claimed is:

1. A skin care active complex comprising:
one or more vitamin A derivatives in an amount of from about 0.001% to about 10% by weight;
one or more hydroxamate derivatives in an amount of from about 0.00001% to about 10% by weight; and
one or more anti-inflammatory natural compound in an amount of from about 0.01% to about 5% by weight.

2. The complex of claim 1 which comprises:
one or more vitamin A derivatives in an amount of from about 0.01% to about 5.0% by weight;
one or more hydroxamate derivatives in an amount of from about 0.0001% to about 0.01% by weight; and
one or more anti-inflammatory natural compounds in an amount of from about 0.1% to about 1% by weight.

3. The complex of claim 1 which comprises:
one or more vitamin A derivatives in an amount of from about 0.01% to about 1.0% by weight;
one or more hydroxamate derivatives in an amount of from about 0.0001% to about 0.01% by weight; and
one or more anti-inflammatory natural compounds in an amount of from about 0.1% to about 1% by weight.

4. The complex of claim 2 further comprising one or more vitamin K in an amount of from about 0.001% to about 10% by weight.

5. The complex of claim 3 further comprising one or more vitamin K in an amount of from about 0.001% to about 10% by weight.

6. The complex of claim 4 wherein the vitamin K is vitamin K-1.

7. The complex of claim 4 wherein the one or more vitamin K is in an amount of from about 0.01% to about 0.1% by weight.

8. The composition of claim 1 wherein the vitamin A derivative is retinol.

9. The composition of claim 1 wherein the vitamin A derivative comprises retinol esters with saturated or unsaturated fatty acids (mono- or di-unsaturated) of chain length from 8-20.

10. The composition of claim 1 wherein the hydroxamate derivative comprises pseudopeptide hydroxamate.

11. The composition of claim 1 wherein the hydroxamate derivative is selected from the group of hydroxamates of salicylic acid and its derivatives, hydrobenzoic acid, tryptophan, amino acids, peptides containing from 1 to 6 amino acids, peptide mimetics, alpha hydroxy acids, dicarboxylic acids, substrate-analogue peptides of matrix metallo proteases containing aminomalonic acid, monohydroxamates of aspartic and glutamic acids.

12. The composition of claim 1 wherein the anti-inflammatory natural compound is ursolic acid.

13. The composition of claim 1 wherein the anti-inflammatory natural compound is selected from the group of vitamin E and its esters, vitamin C and its derivatives, green tea polyphenols, coenzyme Q10, bioflavinoids, plant extracts, pomegranate, amla, and polyphenolic compounds.

14. A cosmetic product comprising from about 1 to about 2% by weight of the complex according to claim 1.

15. A cosmetic product comprising from about 1 to about 2% by weight of the complex according to claim 2.

16. A cosmetic product comprising from about 1 to about 2% by weight of the complex according to claim 4.

17. A sunscreen product comprising the complex according to claim 1 and an effective amount of a sunscreen agent.

18. A sunscreen product comprising the complex according to claim 2 and an effective amount of a sunscreen agent.

19. A sunscreen product comprising the complex according to claim 4 and an effective amount of a sunscreen agent.

20. A method of preventing UV-induced and age-induced wrinkle formation in skin comprising:
formulating an active complex that comprises vitamin A derivatives in an amount of from about 0.001% to about 5% by weight; hydroxamate derivatives in an amount of from about 0.00001% to about 10% by weight; and anti-inflammatory natural compound in an amount of from about 0.01% to about 5% by weight; and optionally, one or more vitamin K in an amount of from about 0.001% to about 10% by weight; and
applying the composition on the skin in one of a cream, lotion and a wash off type product.

21. The method of claim 20 wherein the active complex comprises vitamin A derivatives in an amount of from about 0.001% to about 5% by weight.

22. The method of claim 21 wherein the vitamin K is vitamin K-1; the vitamin A derivative is retinol; the hydroxamate derivative is pseudopeptide hydroxamate;
and the anti-inflammatory natural compound is ursolic acid.

23. A method of improving skin condition comprising:
formulating a composition that comprises vitamin A derivatives in an amount of from about 0.001% to about 10% by weight; hydroxamate derivatives in an amount of from about 0.00001% to about 10% by weight; anti-inflammatory natural compounds in an amount of from about 0.01% to about 5% by weight; and optionally, one or more vitamin K in an amount of from about 0.001% to about 10% by weight; and
applying the composition on the skin in one of a cream, lotion and a wash off type product.

24. The method of claim 23 wherein the active complex comprises vitamin A derivatives in an amount of from about 0.001% to about 5% by weight.

25. The method of claim 24 wherein the vitamin K is vitamin K-1; the vitamin A derivative is retinol; the hydroxamate derivative is pseudopeptide hydroxamate; and the anti-inflammatory natural compound is ursolic acid.

26. A method of manufacturing a skin care active complex comprising combining one or more vitamin K in an amount of from about 0.001% to about 10% by weight; one or more vitamin A derivatives in an amount of from about 0.001% to about 5% by weight; one or more hydroxamate derivatives in an amount of from about 0.00001% to about 10% by weight; one or more anti-inflammatory natural compound in an amount of from about 0.01% to about 5% by weight; and one or more solvents.

27. A method of manufacturing a skin care active complex comprising combining one or more vitamin A derivatives in an amount of from about 0.001% to about 10% by weight; one or more hydroxamate derivatives in an amount of from about 0.00001% to about 10% by weight; one or more anti-inflammatory natural compound in an amount of from about 0.01% to about 5% by weight; and one or more solvents.
